(19) United States
(12) Patent Application Publication
(10) Pub. No.: US 2007/0106226 A1
(43) Pub. Date: May 10, 2007

(54) Syringe with internal sleeve

(76) Inventors: Perry W. Croll, Salt Lake City, UT (US); Mark A. Christensen, Salt Lake City, UT (US); Marshall T. Denton, Salt Lake City, UT (US)

Correspondence Address: TRASK BRITT, P.O. BOX 2550, SALT LAKE CITY, UT, 84110, US

(21) Appl. No.: 11/271,022

(22) Filed: Nov. 9, 2005

(51) Int. Cl. A61M 5/32; A61M 5/00

(52) U.S. Cl. 604/199; 604/110; 604/198

(57) ABSTRACT

An improved syringe with a sleeve operable as a barrier effective to resist entry of undesired particles, such as microorganisms or pathogens, for contact with the inside of the syringe from the plunger end. A portion of the sleeve is configured for disposition surrounding the plunger and inside the syringe's barrel. A syringe structured according to certain principles of the invention is effective to maintain the inside of the syringe's barrel in a sterile condition, even subsequent to multiple strokes of the plunger.

### BACKGROUND OF THE INVENTION

Field of the Invention: This invention relates generally to pumping devices adapted to resist contamination internal to the pump mechanism. In particular, the invention relates to medical syringes.

State of the Art: Syringes are known in the medical field, and typically include a plunger arranged for reciprocal travel inside of a barrel effective alternately either to inspire or eject fluid through a discharge orifice. A stopper carried at an end of the plunger forms a wiping fluid seal against the inside of the barrel. Retracting the plunger causes a vacuum, or pressure lower than ambient, effective to inspire treatment fluid into the barrel. Depressing the plunger causes an increased pressure inside the barrel, higher than ambient, effective to expel the treatment fluid from the barrel.

Syringes are commonly used during medical procedures as pump devices. A typical use for a syringe includes inspiring a treatment fluid into the syringe's barrel through the discharge opening by retracting the plunger, then depressing the plunger to expel the treatment fluid through the discharge opening. Often, a needle, or other device, may be affixed to the discharge end of the syringe to guide or otherwise manipulate the fluid discharged through the syringe's discharge opening. Other times, a syringe may be incorporated into a fluid flow circuit, such as by way of a length of tubing affixed to the syringe's discharge opening.

Multi-use of a syringe entails making a plurality of strokes of a plunger inside a syringe barrel. It is known that undesired particles, such as pathogens or microorganisms, can cross the wiping stopper seal during multiple strokes of the syringe, thereby compromising sterility inside the syringe barrel. Loss of sterility in a closed fluid system can place a patient at risk of nosocomical infection, or line sepsis, such as vascular line sepsis. Any sort of wiping seal, or vented barrier structure, is ineffective to resist entrance of microorganisms from the proximal syringe end for contact with an interior of the syringe barrel. Therefore, in many medical procedures, a syringe is typically removed from its sterile packaging, used only once, and then discarded.

Medical procedures do exist where multi-use of a syringe connected in-circuit at its discharge end to a closed fluid system is desirable. Often, the environment in which the syringe will be placed into service is not sterile, and can even be severely contaminated with pathogens, microorganisms, urine, feces, and other undesired particles. Use of a currently commercially available syringe in such an environment presents serious risk of infecting the patient with undesired particles that enter the syringe's proximal end and cross its wiping seal.

At least four different medical product companies currently offer embodiments of syringes that include protection of some sort to resist entrance of particles into the proximal end of the syringe. Utah Medical Products, of Midvale Utah, offers a closed needleless arterial blood collection kit under the product name Deltran®, which includes a proximally projecting plunger protection sheath. Edwards Lifesciences, LLC, of Irvine Calif. offers a closed injectate delivery system including a proximally projecting contamination shield integrated into the plunger. Becton, Dickinson and Company, of Franklin Lakes, N.J. offers a cardiac output injectate kit under the product number 682260, which includes Touchguard™ sheath for the plunger. And finally, Hospira, of Lake Forrest, Ill. offers a Thermodilution Cardiac Output Injectate System (product invoice THERMOSET—ICED) having a syringe incorporating a Contamination Sheath.

Certain commercially available syringes include a proximally projecting shroud that covers the plunger and is vented to the atmosphere. A vented shroud may advantageously permit sterilization incorporating a vacuum cycle, such as commonly employed with an ETO gas sterilization treatment. However, air inherently flows into, and out of, such a vented shroud during actuation of the plunger. The air flowing into a syringe through a vented shroud will inevitably transport undesired particles into the interior of the syringe. Therefore, all such vented syringes lack a truly effective hermetic proximal seal or barrier.

Commercially available shrouds often are formed from silicone material. Unfortunately, such silicone material has an affinity for dirt, or contaminants. A proximally projecting shroud presents a sizable exposed surface of material that can be characterized as a “dirt magnet.” The presence of a large exposed silicone surface can frustrate effective cleaning of the local environment surrounding that surface.

By experimentation, Applicants have determined that an unvented, proximally projecting shroud can sometimes interfere with actuation of the plunger by “ballooning” or inflating as the plunger is depressed toward a fully discharged position. The puffed-up portion of such an inflated shroud can interfere with the plunger's stroke, or create difficulty in gripping the plunger for retraction from a depressed position. The inflation problem is exacerbated in situations where the shroud volume (defined between a retracted shroud and plunger) is larger than the receiving volume available inside a barrel at a plunger-depressed position. Therefore, simply removing, or sealing, the shroud vent may not produce a desirable, hermetically sealed syringe.

There are disadvantages associated with handling a syringe's plunger indirectly through a proximally projecting external “boot” or shroud arranged to cover the plunger. Such a shroud generally interferes to obfuscate a user's tactile senses and thereby reduce feedback as the user manipulates the plunger. Shrouds, or rearwardly projecting “boots” are particularly intrusive and cumbersome when the plunger is substantially depressed. At such position, the shroud covering the plunger typically is bunched-up, or wrinkled. The bunched-up portion of shroud material can interfere with the user obtaining a sufficient purchase on the plunger to effect a retracting motion of the plunger.

Certain advances in the art of protecting proximal ends of syringes against contaminants are disclosed in US patents, including U.S. Pat. No. 6,830,564 to Gray, and U.S. Pat. No. 4,713,060 to Riuli. The entire disclosures of the aforementioned patents are hereby incorporated as though set forth herein in their entireties as background related to syringe construction. In the latter '060 patent, Riuli discloses a flexible cover arranged to project proximally from the syringe's barrel and to cover the plunger. However, as illustrated in FIG. 4, the flexible cover is bunched-up at the fully depressed plunger. As previously discussed, such bunched-up cover material disadvantageously can make the plunger difficult to grasp.

In the former '564 patent, Gray discloses several embodiments of syringes adapted in various ways to reduce entrance of contaminants into the syringe barrel cavity. Certain of Gray's embodiments include proximally projecting coverings for plunger structure which are arranged to fold at selected hinging locations. Other embodiments include proximally projecting covers arranged to cover the plunger and add one or more wiping seals, or provide a labyrinth barrier to particles at the barrels proximal end. One embodiment provides a proximal end seal arranged to wipe the shaft of the plunger. The disclosed embodiments are either expensive and difficult to manufacture, cumbersome to use, and/or fail to provide adequate resistance to undesired particles entering the barrel cavity from the proximal end.

It would be an improvement to provide a syringe including a sealing arrangement, for its proximal end, which is truly effective to resist contamination of the inside of the syringe from its proximal end. Such an improved syringe would then be capable of sustaining multiple-use in closed systems, in even a non-sterile medical environment, without placing the patient at risk of syringe-induced contamination. It would be a further improvement to provide a syringe having a hermetically sealed proximal end that does not interfere with operation of the syringe's plunger.

### BRIEF SUMMARY OF THE INVENTION

The invention may be embodied as a syringe, including a barrel, a plunger and a sleeve. The barrel may be constructed in conventional fashion to extend along an axis between a proximal end and a distal end, and to have a discharge opening associated with the distal end. The plunger is conventionally arranged for reciprocal actuation of a stopper along the axis of the barrel. The sleeve is typically disposed between a shaft portion of the plunger and the barrel to cause distally directed motion of a portion of the sleeve inside the barrel as the plunger is actuated in a direction effective to cause a discharge from the syringe. A sleeve typically includes a tubular barrier element arranged to resist contact between a portion of an inside surface of the barrel and undesired particles attempting to enter the syringe from a proximal end of the barrel. Preferably, the sleeve is adapted to form a hermetic seal, for an interior working portion of the barrel, against intrusion of undesired particles from a proximal end of the syringe.

In general, a perimeter of the sleeve is anchored to structure associated with the barrel effective to form a sealed interface. Such sealed interface provides a proximal barrier around a perimeter of the barrel to resist contamination of the inside of the syringe by undesired particles attempting to enter the syringe from its proximal end. Sometimes, a sleeve is anchored to, and/or by, structure associated with a proximal end of the barrel. One operable sleeve may be characterized as a sock forming an uninterrupted barrier established at a sealed interface around a perimeter of a proximal end of the barrel. Certain sleeves are sized in length such that a distally directed motion of the sleeve causes a resilient extension of a tubular portion of the sleeve when the syringe is in a fully discharged configuration.

In one currently preferred embodiment, a plunger includes a shaft adapted at its distal end to carry a stopper. The sleeve is configured as an uninterrupted sock having a toe carried at a distal end of a tube, with a portion of the sock being disposed between cooperating portions of the plunger's shaft and the inside of a barrel of an assembled syringe. The stopper may be configured to trap the toe of the sock between the stopper and the distal end of the plunger's shaft.

In any case, a syringe structured according to certain principles of the invention includes a sleeve having a portion adapted for reciprocal motion inside a portion of the syringe barrel. An exemplary sleeve includes a tube element structured to form an axially extending barrier. A distal length of such tube is extendable into the barrel for disposition between a distal portion of the plunger's shaft and a working portion of the barrel. A distal portion of the tube is configured in harmony with the shaft to form a distal barrier. A proximal portion of the tube can be anchored to structure associated with the barrel effective to form a proximal perimeter barrier. In combination, the distal barrier, the axially extending barrier, and the proximal perimeter barrier are structured to resist contact between undesired particles, entering through the proximal end of the barrel, and an inside surface of the working portion of the syringe's barrel.

Sometimes, a syringe may also include anti-removal structure to resist accidental retraction of the plunger from engagement inside the barrel. One arrangement forming operable anti-removal structure includes a radially protruding rim carried proximate a distal end of the plunger. A circumference of that rim desirably has an outside diameter sized in harmony with an inside diameter of a ring carried by the barrel to form an annulus in which to accommodate a thickness of the sleeve in a sufficiently close fit to provide anti-removal feedback for a plunger in the barrel while still permitting assembly of the plunger into the barrel, and repetitive actuation of the syringe, without causing undue damage to the sleeve.

Certain embodiments of the invention may also include anti-rotation structure arranged to resist rotation of the plunger about a syringe's axis. Embodiments of syringes according to the invention may optionally include sleeve containment structure arranged to resist egress of a sleeve from substantial confinement inside the syringe's barrel. Sometimes, anti-rotation structure may additionally operate, in-part, as sleeve containment structure. Also, anti-rotation structure can be arranged to additionally resist accidental withdrawal of the plunger from an installed position inside the barrel.

### BRIEF DESCRIPTION OF THE SEVERAL VIEWS OF THE DRAWINGS

In the drawings, which illustrate what are currently considered to be the best modes for carrying out the invention:

FIG. 1 is an exploded assembly plan view of a first embodiment constructed according to certain principles of the instant invention;

FIG. 2 is a cross-section view taken through a midline along an axis of the embodiment illustrated in FIG. 1 in a first assembled position;

FIG. 3 is a plan view of the embodiment illustrated in FIG. 1 in a second assembled position;

FIG. 4 is an end view of one embodiment of structure arranged to resist rotation of a plunger installed in a syringe;

FIG. 5 is a plan view, partially in section, of a second embodiment of a syringe constructed according to certain aspects of the invention; and

FIG. 6 is a plan view, partially in section, of a portion of a third embodiment of a syringe constructed according to certain aspects of the invention.

### DETAILED DESCRIPTION OF THE ILLUSTRATED EMBODIMENTS

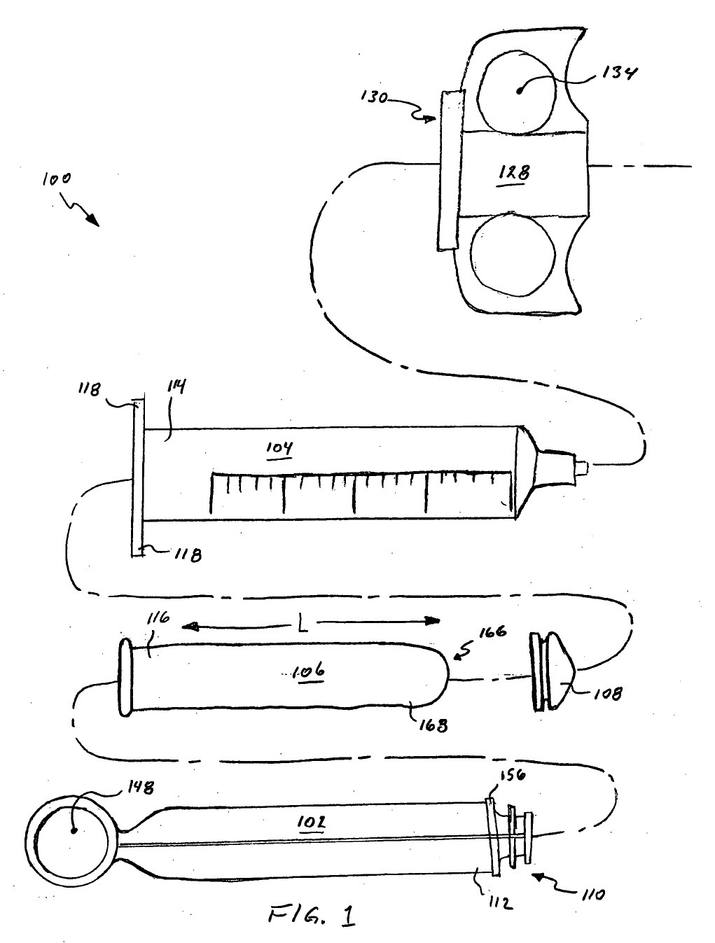

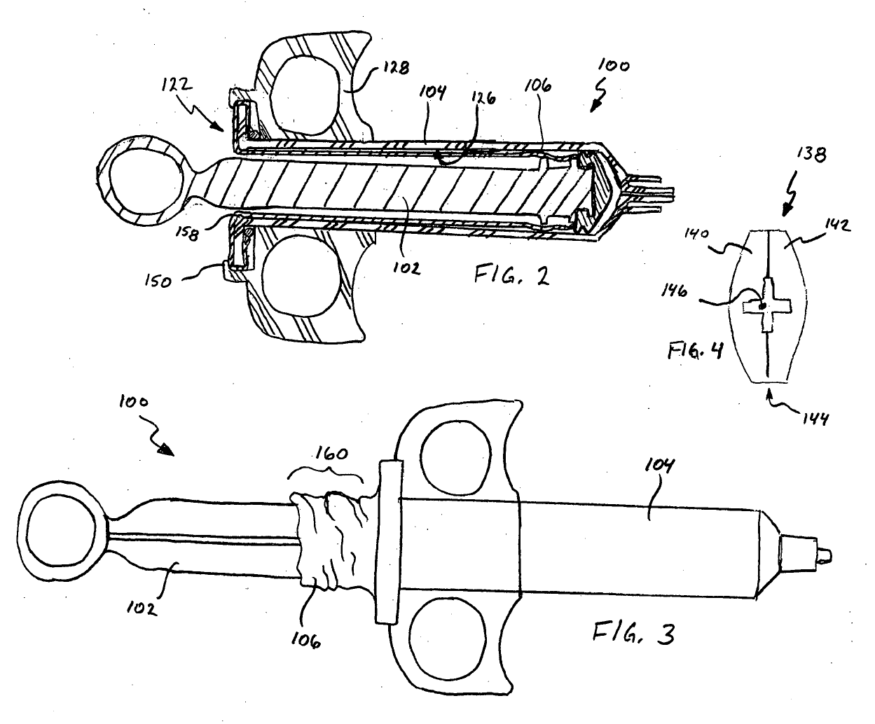

A first embodiment of an improved syringe constructed according to certain principles of the invention is indicated generally at 100 in FIGS. 1 through 3. With particular reference to FIG. 1, syringe 100 includes a plunger 102 sized for reciprocal actuation inside barrel 104. In the illustrated embodiment 100, plunger 102 is first inserted into a sleeve 106. Then stopper 108 is secured over sleeve 106 in engagement with holding structure, generally indicated at 110, carried at distal end 112 of plunger 102. The resulting assembly is then inserted into barrel 104 from its proximal end 114. A proximal end 116 of sleeve 106 may then be anchored to barrel 104 to form a sealed interface. As best illustrated in FIG. 2, one operable anchoring arrangement includes stretching proximal end 116 of sleeve 106 over ears 118 to form a proximal barrier, generally indicated at 122.

The proximal perimeter barrier 122, formed between the sleeve 106 and the barrel 104, is formed completely around a perimeter of the barrel 104. Furthermore, the proximal barrier is typically arranged to form a static seal. By static seal, it is meant that no relative motion between components at the sealing barrier is required. An effective static seal may be characterized as a hermetic seal. Desirably, the proximal seal 122 is a hermetic seal effective to resist entrance of undesired particles, such as pathogens or microorganisms, into the barrel 104 (from its proximal end 114), for contact with its inside working surface, generally indicated at 126 in FIG. 2. Working surface 126 may be defined as that portion of barrel 104 that is wiped, or contacted, by stopper 108 during conventional actuation of syringe 100.

With particular reference to FIGS. 1 and 2, in some cases, the barrel 104 with its installed sleeve 106 may be inserted into optional auxiliary finger support or finger loop structure 128. It is within contemplation that optional finger structure 128 can include socket structure, generally indicated at 130, configured to cause an additional clamping force effective to reinforce the seal formed at proximal perimeter barrier 122. It is further within contemplation that such reinforcement structure may be provided as an alternatively configured socket 130, such as simply socket 130 without including additional structure arranged to form finger holes 134.

In some situations, it may be desirable to resist rotation of a plunger 102 that is installed in a barrel 104. For example, the sleeve 106 may suffer damage due to excessive twisting about an axis of the barrel 104. As one way to resist such damage, certain syringes may include structure configured to resist rotation of an installed plunger 102. One such anti-rotation structure is illustrated in FIG. 4, and is generally indicated at 138.

The illustrated anti-rotation structure 138 may be characterized as a clamshell plate having a substantially constant thickness in a direction into the page. Left side 140 and right side 142 are rotatably connected at hinge 144. In the closed position illustrated, an opening 146 is defined in which to receive an axially extending shaft portion of a plunger, such as plunger 102, for reciprocal actuation of the plunger in a barrel. The illustrated opening 146 provides structure configured in harmony with the plunger 102 effective to interfere with radially protruding structure of the plunger, thereby to resist rotation of the plunger. The left side 140 and right side 142 can be swung open about hinge 144, and subsequently closed to surround a shaft portion of plunger 102 disposed within opening 146.

Opening 146 may be arranged to permit a limited amount of rotation of a plunger 102. Sometimes, it is desirable to permit a plunger to rotate a certain amount during actuation of a syringe. For example, in use of a syringe having a thumb hole 148 in plunger 102 (e.g., as illustrated in FIG. 1), a user may feel more comfortable if the plunger 102 is permitted to rotate a certain amount as it travels along an axis of barrel 104. Also, a certain amount of rotation of plunger 102 may be desirable to accommodate both left- and right-handed use of a syringe.

A closed anti-rotation structure 138 (or assembled components of anti-rotation structure), may conveniently be retained in a closed position inside a socket 130, disposed proximally on top of an installed barrel 104. Walls of the socket 130 can be arranged to resist an opening rotation of left side 140 and right side 142 about hinge 144. As illustrated, anti-rotation structure 138 is maintained inside socket 130 by an installed capture behind one or more cantilevered arms 150. Other known retention structure is workable, including friction fits between components, other arrangements causing a structural interference, adhesives, welding, heat staking, or other conventional fastening arrangements. An installed anti-rotation structure 138 may also be structured and arranged to cause a reinforcing clamping force on a proximal perimeter barrier 122 to further resist entrance of undesired particles into the barrel 104.

Commercially available syringes typically include structure arranged to resist accidental extraction of the plunger from inside the barrel. In certain such syringes, a radially protruding rim 156 (see FIG. 1) is structured in harmony with a cooperating barrel ring 158 disposed to protrude radially inward adjacent the proximal entrance of barrel 104. A user of such commercially available syringe obtains feedback in the form of increased resistance to further retraction of the plunger as the rim 156 and barrel ring 158 come into contact at a fully retracted position. Such an arrangement of anti-separation structure is also operable in syringes according to the instant invention, although it is currently preferred to size the rim 156 and barrel ring 158 to accommodate the sleeve 106 for repetitive actuation without imparting damage to the sleeve. Anti rotation structure 138 is also one example of alternative structure effective to resist accidental complete extraction of a plunger 102 from engagement inside barrel 104.

In certain syringes structured according to the instant invention, an annulus between a rim 156 and a barrel ring 158 is desirably provided in which to receive the thickness of the sleeve 106. Such annulus may be sized to facilitate assembly of a plunger 102 and sleeve 106 into a barrel 104, and still provide anti-removal feedback at a fully retracted plunger position. Also, the portions of the rim 156 and barrel ring 158 which contact the sleeve 106 desirably include sufficiently large radii to resist formation of a stress concentration pressure point in the sleeve wall, and corresponding damage or reduction in useable life of a syringe's sleeve 106.

Sometimes, it is desirable to construct the various components of a syringe from materials sized to provide substantially the same “feel” to a user as is provided by commercially available syringes. The “feel” includes the force necessary to depress or retract a plunger 102 with respect to the barrel 104. The sizes, and materials of construction, of a rim 156, sleeve 106, stopper 108, and inside surface 126 may be adjusted to create substantially the same “feel” as commercially available syringes lacking an effective proximal end barrier.

However, sometimes it is desirable to structure a syringe to take advantage of the disparity between strength of a user's hand in a syringe discharge direction, compared with the reduced strength of the user's hand in a syringe filling direction. In such case, a resilient sleeve 106 may be structured to be shorter in a length direction “L” than a barrel 104. Such a “short” sleeve 106 therefore assists in retracting the plunger 102 to fill the syringe. The additional force to axially “stretch” such a sleeve 106 to discharge treatment fluid from a syringe can easily be accommodated by the user's superior strength of motion in the discharge direction.

In certain cases, socket structure 130 may be configured to define an axially extending compartment in which to hold an everted portion 160 (see FIG. 3) of sleeve 106, thereby to resist contact between the sleeve 106 and potentially damaging objects external to the syringe. Anti-rotation structure 138, or an alternatively structured aperture having a close-fit to the plunger 102, can trap, or confine, the sleeve 106 inside the barrel 104 or socket 130. Anti-rotation structure 138 may alternatively be structured to help define an axially extending cavity in which to confine a sleeve 106. In addition to providing an enhanced safety aspect, a syringe structured to hold the sleeve 106 substantially inside the barrel 104 or in a socket, such as socket 130, may also provide a more pleasing appearance.

A workable sleeve 106 maybe manufactured from material capable of being formed into a membrane effective as a barrier to resist undesired particles. One effective sleeve 106 can be characterized as a condom. A currently preferred sleeve material for certain medical applications includes Nitrile. It is within contemplation for a sleeve to be formed from polymerized plastic material, such as commonly available poly bag material. Latex is an operable sleeve material, although not currently preferred due to its potential for causing allergic reactions in certain patients. Sleeves 106 may be formed using a variety of known manufacturing techniques, including extrusion, injection molding, casting, and dip molding or spray application on a male mandrel.

Preferred sleeves 106 include a substantially cylindrical length portion disposed between proximal and distal ends. Such length portion forms an axially extending barrier against undesired particles. One currently preferred sleeve 106 (e.g., see FIGS. 1-3), is configured to provide an uninterrupted barrier that can be sealed with respect to a barrel 104 at its proximal end. Such sleeve 106 may be characterized as an approximately tubular sock having a toe, generally indicated at 166, disposed at its distal end 168. Toe 166 inherently forms one embodiment of a distal barrier to entrance of undesired particles into the syringe. Anchoring the perimeter of such sock to structure associated with a barrel 104 can form a hermetically sealed interface around a perimeter of the barrel 104 and provide a total barrier to resist entrance of pathogens, or undesired particles, from the proximal end of a syringe. An inside surface of an installed sleeve 106 maybe defined as the surface adjacent to surface 126 of barrel 104. Undesired particles may contact the exterior surface of the sock, and may even technically be drawn “into” the barrel 104 as a distal portion of the sock moves distally inside the barrel. However, the undesired particles cannot cross the barrier, formed by the sock, to contact the inside working surface 126 of the barrel.

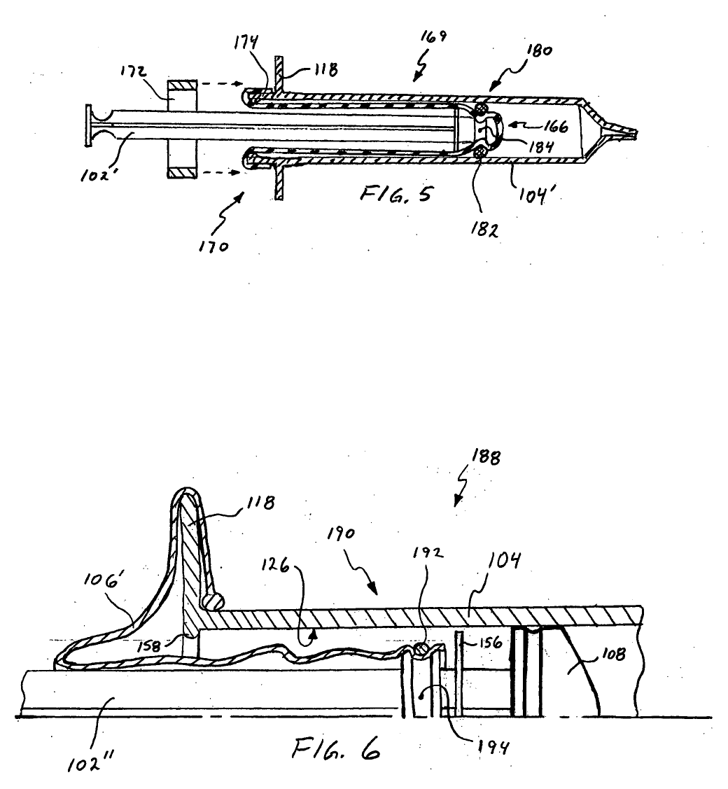

The embodiment 100 in FIGS. 1-3 illustrates a sleeve 106 in association with a syringe that is substantially a stock commercially available syringe. Such an arrangement provides an advantage by incorporating a minimal number of additional components with certain commercially mass-produced, and low cost, components. However, it is within contemplation to make additional modifications to certain portions of a syringe to form alternative embodiments structured according to principles of the instant invention. Certain of such modifications are illustrated in FIGS. 5 and 6.

FIG. 5 illustrates an alternative syringe, generally indicated at 169, constructed according to certain principles of the instant invention. Syringe 169 includes an alternative proximal anchoring arrangement for a sleeve 106, generally indicated at 170. Anchor 170 includes a ring 172 structured to clamp an everted proximal portion of sleeve 106 against a cylindrical extension 174 of barrel 104′. Such ring 172 may be self-biased to exert a radial clamping force. It is within contemplation for a ring 172 to be structured alternatively to trap a proximal portion of a sleeve against an inside cylindrical surface of a barrel 104 or 104′. In such case, the alternative ring may also operate to resist unintentional retraction of a plunger from within the barrel. Also, an alternatively structured ring may operate to contain the sleeve 106 inside the barrel, or other protective area. Furthermore, an alternatively structured ring may include anti-rotation structure to resist rotation of an installed plunger.

FIG. 5 also illustrates an alternative stopper arrangement, generally indicated at 180, that forms an alternative distal perimeter seal and distal anchor arrangement. A resilient ring 182, such as an O-ring, is engaged in a receiving trough 184 carried on distal end of plunger 102′. Such ring 182 represents an alternative structure effective to affix, or anchor, a distal portion of sleeve 106 to a plunger 102′. Ring 182 desirably forms a static distal perimeter seal disposed between the sleeve 106 and trough structure in plunger 102′. Such a static perimeter seal serves as an effective back-up seal to the primary distal seal provided by the uninterrupted membrane forming toe 166 (which is an absolute barrier to undesired particles). Additionally, illustrated ring 182 forms a separate wiping seal in cooperation with working surface 126 to control inspiration and discharge of treatment fluids.

FIG. 6 illustrates a portion of another alternative syringe, generally indicated at 188, constructed according to certain principles of the instant invention. Syringe 188 includes a second alternative distal perimeter seal arrangement, generally indicated at 190. Distal perimeter seal 190 includes a resilient ring 192, such as an O-ring, disposed to compress the perimeter of an open distal portion of sleeve 106′ against the circumferentially extending surface of trough 194 carried at a distal end of plunger 102′. Therefore, distal perimeter seal 190 is a static seal effective to resist entrance of undesired particles into the syringe for contact with surface 126.

The anti-removal rim 156, barrel ring 158, and stopper 108 may be configured identically to corresponding components of a comparable commercially available syringe. Desirably, trough 194 is disposed proximal to anti-removal rim 156, as illustrated. Such positioning eliminates pinching contact of the sleeve 106′ between surface 126 and/or barrel rim 158 effective to enhance robustness and working life of the sleeve 106′. Furthermore, in such a syringe 188 having such a configuration, the “feel” during actuation of the syringe may be substantially the same as that produced in a comparable syringe lacking any proximal barrier.

Embodiments of the invention may be constructed and arranged such that, at a plunger-depressed position, the internal sleeve 106, 106′ is disposed substantially parallel to and congruent with, the inner wall forming the barrel. Therefore, very little air is trapped between such a sleeve and the working surface of the barrel. Such an arrangement forms a device capable of withstanding a vacuum cycle, such as associated with ETO sterilization, without the potential for sheath swelling, and potential rupture, associated with an unvented external sheath.

What is claimed is:

1. A syringe, comprising:
a barrel, extending along an axis between a proximal end and a distal end, having a discharge opening associated with said distal end;
a plunger arranged for reciprocal actuation along said axis; and
a sleeve between said plunger and said barrel, said sleeve being associated with a shaft portion of said plunger to cause distally directed motion of a portion of said sleeve inside said barrel as said plunger is actuated to a fully discharged position.

2. The syringe according to claim 1, wherein:
said sleeve comprises a tubular barrier element arranged to resist contact between a portion of an inside surface of said barrel and undesired particles attempting to enter said syringe from a proximal end of said barrel.

3. The syringe according to claim 2, wherein:
a perimeter of said sleeve is anchored to structure associated with said barrel effective to form a sealed interface.

4. The syringe according to claim 3, wherein:
said sealed interface provides a proximal perimeter barrier to said undesired particles, said proximal perimeter barrier being established around a perimeter of said barrel.

5. The syringe according to claim 2, wherein:
said sleeve is anchored to structure associated with a proximal end of said barrel.

6. The syringe according to claim 1, wherein:
said sleeve comprises a sock forming an uninterrupted barrier established around a perimeter of a proximal end of said barrel, said barrier being effective to resist contact between a portion of an inside surface of said barrel and undesired particles attempting to enter said syringe from a proximal end of said barrel.

7. The syringe according to claim 1, wherein:
said distally directed motion of said sleeve causes a resilient extension of a tubular portion of said sleeve when said syringe is in a fully discharged configuration.

8. The syringe according to claim 1, wherein:
said sleeve is adapted to form a hermetic seal, for an interior working portion of said barrel, against intrusion of undesired particles from a proximal end of said syringe.

9. The syringe according to claim 1, wherein:
said plunger comprises a shaft adapted at its distal end to carry a stopper;
said sleeve is configured as an uninterrupted sock having a toe carried at a distal end of a tube, a portion of said sock being disposed between cooperating portions of said shaft and said barrel of an assembled syringe; and
said stopper is configured trap said toe of said sock between said stopper and said distal end of said shaft.

10. The syringe according to claim 1, wherein:
said plunger comprises a shaft adapted at its distal end to carry a stopper;
said sleeve comprises a tube structured to form an axially extending barrier; with
a distal length of said tube being disposable between a distal portion of said shaft and a working portion of said barrel;
a distal portion of said tube being configured in harmony with said shaft to form a distal barrier; and
a proximal portion of said tube being anchored to structure associated with said barrel effective to form a proximal perimeter barrier; wherein:
said distal barrier, said axially extending barrier, and said proximal perimeter barrier are structured to resist contact between undesired particles, entering through said proximal end of said barrel, and an inside surface of said working portion of said barrel.

11. The syringe according to claim 1, further comprising:
a radially protruding rim carried proximate a distal end of said plunger, a circumference of said rim having a outside diameter sized in harmony with an inside diameter of a ring carried by said barrel to form an annulus in which to accommodate a thickness of said sleeve in a sufficiently close fit to provide anti-removal feedback for said plunger in said barrel while still permitting repetitive actuation of said syringe.

12. The syringe according to claim 1, further comprising:
anti-rotation structure arranged to resist rotation of said plunger about said axis.

13. The syringe according to claim 1, further comprising:
sleeve containment structure arranged to resist egress of said sleeve from substantial confinement inside said barrel.

14. The syringe according to claim 1, further comprising:
plunger containment structure arranged to resist accidental withdrawal of said plunger from an installed position inside said barrel.

15. A syringe that is capable of multi-use in a medical setting, the syringe comprising:
a barrel, extending along an axis between a proximal end and a distal end, having a discharge opening associated with said distal end;
a plunger having a portion arranged for reciprocal actuation inside said barrel along said axis; and
a barrier element associated with said plunger and structured as a hermetic seal effective to resist entrance of microorganisms from said proximal end into an interior of said barrel, for contact with a working surface of said barrel by said microorganisms, as said syringe is actuated to a fully discharged position, said barrier element being configured and arranged to permit a user to directly contact a proximal portion of said plunger for actuation of said syringe.

16. The syringe according to claim 15, wherein:
said barrier element comprises a sleeve disposed between a shaft portion of said plunger and said barrel to permit distally directed motion of a portion of said sleeve inside said barrel as said plunger is actuated in a direction effective to cause a discharge from said syringe.

17. The syringe according to claim 15, wherein:
said barrier element is configured and arranged to provide a hermetic seal between an interior working surface of said barrel and a proximal end of said syringe.

18. The syringe according to claim 15, wherein:
said barrier element comprises a tubular sleeve affixed at its distal end to structure associated with said plunger, a proximal end of said sleeve being anchored around its perimeter to structure associated with a proximal end of said barrel.

19. The syringe according to claim 18, wherein:
said tubular sleeve is affixed at its distal end to structure associated with a distal end of said plunger.

20. The syringe according to claim 18, wherein:
said plunger comprises a shaft adapted at its distal end to carry a stopper;
said sleeve is configured as an uninterrupted sock having a toe at its distal end, a portion of said sock being disposable between cooperating portions of said shaft and a working portion of said barrel of an assembled said syringe.
